(19) United States
(12) Patent Application Publication
(10) Pub. No.: US 2023/0248087 A1
(43) Pub. Date: Aug. 10, 2023

(54) NIPPLE PATCH

(71) Applicant: Yoon Young NA, Goyang-si (KR)

(72) Inventor: Yoon Young NA, Goyang-si (KR)

(21) Appl. No.: 17/981,976

(22) Filed: Nov. 7, 2022

(30) Foreign Application Priority Data
Feb. 9, 2022 (KR) 10-2022-0016701

(51) Int. Cl. A41D 13/05; A61F 13/14

(52) U.S. Cl. CPC A41D 13/0518; A61F 13/143

(57) ABSTRACT

Provided is a nipple patch for covering a male nipple. The nipple patch includes a spider film with an orb web structure to enhance nipple coverage and a finishing film to maximize the coverage of the nipple patch. In addition, the nipple patch provides a soft feel, enables the skin to move freely, and can be easily attached to skin around the nipple. The nipple patch includes a shield covering and protecting a nipple, a flexible adhesive film having adhesion to the skin around the nipple and having a center portion to which the shield is attached, and a spider film attached to a rear surface of the adhesive film, having lower flexibility than the adhesive film, and having an orb web structure to prevent the adhesive film from being stretched.

### BACKGROUND OF THE DISCLOSURE

### 1. Field of the Disclosure

The present disclosure relates to a nipple patch for covering a male nipple. More particularly, the present disclosure relates to a nipple patch including a spider film and a finishing film. The spider film has an orb web structure to increase nipple coverage. The finishing film maximizes the coverage of the nipple patch. In addition, the nipple patch provides a soft feel, enables the skin to move freely, and can be easily attached to the skin around the nipple.

### 2. Description of the Related Art

In order to prevent nipples from showing through when wearing a top, Korean Patent No. 10-2140954 on “NIPPLE-COVERING BAND FOR MEN”, Korean Patent No. 10-2333740 on “Nipple Patch”, and the like have been disclosed as a nipple patch for covering a male nipple in the related art.

“NIPPLE PATCH” of Korean Patent No. 10-2333740 has a dual structure composed of a shield and a main body. The shield is attached to a center portion of the main body and covers the nipple, and the main body adheres to skin around the nipple. For reference, “NIPPLE-COVERING BAND FOR MEN” of Korean Patent No. 10-2140954 also has a dual structure composed of a band and a coating layer. The band is attached to a center portion of the coating layer and covers a nipple.

In Korean Patent No. 10-2333740 on “Nipple Patch”, the flexibility of the main body is required to be low to firmly press the nipple so that the nipple is prevented from showing through a top. On the other hand, the flexibility of the main body is required to be high so that the irritation of the skin to which the nipple patch is attached and the foreign body sensation is reduced. In addition, high flexibility enables the skin to move freely.

The existing nipple patch having the dual structure can be provided with a characteristic of either nipple coverage (preventing a nipple from showing through) or skin adaptability (reducing skin irritation, providing a skin-like feel, and enabling skin to move freely), but not both, according to the flexibility of the main body.

### SUMMARY OF THE INVENTION

The present disclosure is proposed to solve such problems of the nipple patch in the related art. An objective of the present disclosure is to provide a nipple patch having a triple structure or a quadruple structure, thereby having excellent nipple coverage as well as skin adaptability

In addition, another objective of the present disclosure is to provide a nipple patch remaining unfolded when removed from a release film. As a result, the nipple patch can be easily and quickly attached without wrinkles and curves when attached to a nipple and skin around the nipple. Furthermore, an anti-folding member is deactivated once the nipple patch is completely attached to the skin. Therefore, the nipple patch is easily deformable, thereby providing a soft feel and enabling the skin to move freely.

In order to achieve the above objective, the nipple patch, according to the present disclosure, includes a shield covering and protecting a nipple, a flexible adhesive film having an adhesion to skin around the nipples and including center portion to which the shield is attached. In addition, the nipple patch further includes a spider film attached to a rear surface of the adhesive film. The spider film has a lower flexibility than the adhesive film and has an orb web structure to prevent the adhesive film from being stretched.

In addition, the nipple patch further includes a finishing film being attached to a rear surface of the spider film. The finishing film has a lower flexibility than the adhesive film to firmly press the nipple.

In addition, the adhesive film includes an anti-folding member on a frontal surface thereof and an adhesive layer covering the anti-folding member, the anti-folding member being made of a material soluble at body temperature.

According to the present disclosure configured as described above, a nipple patch includes an adhesive film, a spider film, and a finishing film. The adhesive film is highly flexible, thereby having excellent skin adaptability. The spider film has pulling function to prevent the adhesive film from being excessively stretched. As a result, the nipple patch can firmly hold and press a nipple and skin around the nipple, thereby sufficiently covering the nipple. The finishing film holds the spider film and the adhesive film to maximize the coverage of the nipple patch. In addition, the nipple patch remained unfolded when removed from a release film. Therefore, the nipple patch is easily attachable without wrinkles and curves when attached to the nipple and skin around the nipple. Furthermore, an anti-folding member is deactivated once the nipple patch is completely attached to the skin. As a result, the nipple patch is easily deformable, thereby providing a soft feel and enabling the skin to move freely.

### BRIEF DESCRIPTION OF THE DRAWINGS

FIG. 1 is a perspective view and a cross-sectional view of a nipple patch according to the present disclosure;

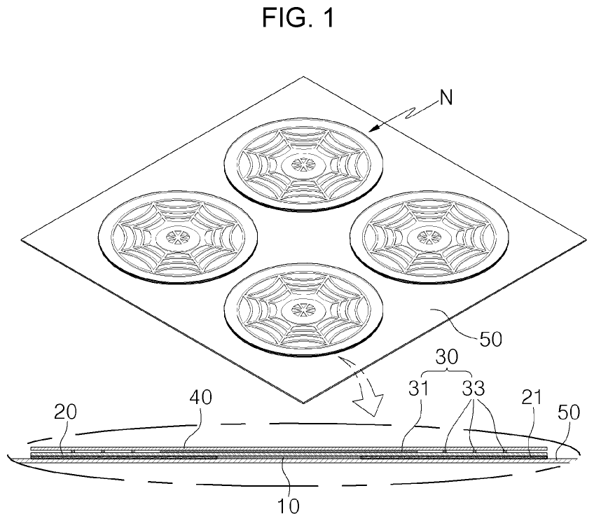

FIG. 2 is an exploded perspective view of the nipple patch according to FIGS. 1; and

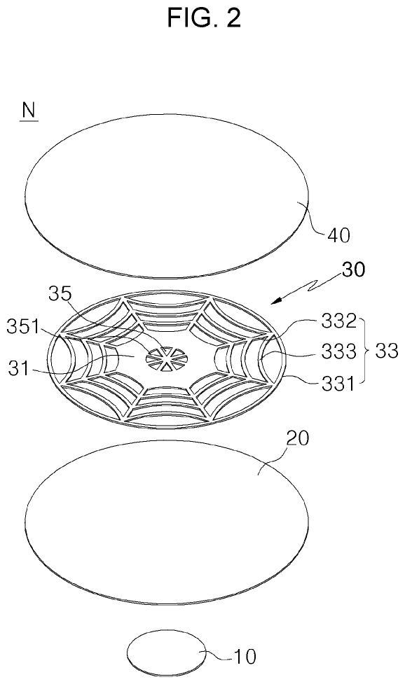

FIG. 3 is a diagram illustrating an adhesive film including an anti-folding member.

### DESCRIPTION OF THE PREFERRED EMBODIMENTS

As shown in the drawings, a nipple patch N, according to the present disclosure, includes a shield 10, an adhesive film 20, a spider film 30, and a finishing film 40. Furthermore, the nipple patch N includes a release film 50 for the protection of the adhesive film 20 during the production and distribution of the nipple patch N.

The shield 10 covers and protects a sensitive nipple, and intensively presses the nipple.

The shield 10, which is in direct contact with the nipple of a user, may be made of a non-woven fabric, a synthetic resin, paper, a composite material, and the like having non-adhesion to reduce skin irritation and foreign body sensation.

The adhesive film 20 adheres to the skin around the nipple, holds the skin, including the nipple and areola, together, and firmly presses the same.

The shield 10 is attached to a central portion of a frontal surface of the adhesive film 20. On the frontal surface of the adhesive film 20, an adhesive layer 21 to which the skin is attached is formed. Preferably, the adhesive layer 21 has strong adhesion so that the nipple patch N does not come off unintentionally from the attached skin. The adhesive layer 21 may be formed by coating an adhesive or attaching a double-sided tape onto the adhesive film 20.

A film such as PVU, which is deformable, soft and highly flexible, is used as the adhesive film 20, so that the skin being in contact with the adhesive film 20 has excellent adaptability. Hence, the skin in contact with the adhesive film 20 has low irritation and foreign body sensation, and is enabled to move freely.

The spider film 30 firmly holds and presses the nipple and areola. In addition, the spider film 30 prevents the adhesive film 20 from being stretched to increase the nipple coverage. Furthermore, the spider film 30 connects the adhesive film 20 with the finishing film 40 so that the nipple coverage is maximized.

The spider film 30 is attached to a rear surface of the adhesive film 20.

The spider film 30 includes a pressing portion 31 and an elongation-inhibiting portion 33. The pressing portion 31 is disposed on an inner portion of the spider film 30 and has a blocked form to hold and press the nipple and the areola of the user. The elongation-inhibiting portion 33 is disposed on an exterior portion of the spider film 30 and has an orb web structure to prevent the adhesive film 20 from being stretched.

The orb web structure of the elongation-inhibiting portion 33 is composed of an outermost circle 331, a plurality of straight lines 332 toward the center of the outermost circle 331, and a plurality of arc lines 333 connecting every two adjacent straight lines 332 with curve.

The center of the pressing portion 31 is formed of a plurality of extended lines 351, which are the lines extended from the plurality of the straight lines 332. As a result, a center-indicating portion 35 is provided to inform the center of the nipple patch N so that the nipple is positioned thereto.

The spider film 30 has lower flexibility than the adhesive film 20 to firmly hold and press the nipple and areola of the user, thereby increasing the nipple coverage. In addition, the spider film 30 has the orb web structure to reduce skin irritation and foreign body sensation and enable the skin to move freely, thereby enhancing skin adaptability.

More specifically, the pressing portion 31 of the spider film 30 has lower flexibility than the adhesive film 20. As a result, the nipple and areola is firmly held and pressed, thereby increasing the nipple coverage. In addition, the elongation-inhibiting portion 33 has the orb web structure.

Therefore, the spider film 30 has higher contractility and lower elongation than the adhesive film 20, thereby enhancing the skin adaptability. The elongation-inhibiting portion 33 has low elongation so that the adhesive film 20 is prevented from being excessively stretched. In addition, the elongation-inhibiting portion 33 has high contractility so that the adhesive film 20 does not interfere with restoration after contraction. Furthermore, the elongation-inhibiting portion 33 allows the adhesive film 20 to have high flexibility to enhance the skin adaptability.

The spider film 30 may be provided by being printed on a rear surface of the adhesive film 20, or being coated with an adhesive.

The finishing film 40 maximizes the coverage of the nipple patch and smooths an outer surface thereof to be exposed.

The finishing film 40 is attached to a rear surface of the spider film 30.

The finishing film 40 has lower flexibility than the adhesive film 20. As a result, the strength of holding and pressing the nipple and areola of the user is enhanced, thereby maximizing the coverage of the nipple patch. For such a finishing film 40, a PU film may be used. The PU film has good deformability, thereby providing a soft feel. In addition, the PU film enables the skin to which the nipple patch N is attached to move freely.

The release film 50 covers and protect the adhesive layer 21 in the adhesive film 21 during the production, transportation before using, and storage. When the nipple patch N is used, the release film 40 may be removed from the adhesive film 20 and allow the adhesive layer 21 to adhere to the skin.

A plurality of the nipple patches N may be attached to a single sheet of the release film 50.

In the nipple patch N, the shield 10 and the spider film 30 covering the nipple of the user preferably have a pink-tone color to be similar to the skin color. In addition, the adhesive film 20 and the finishing film 40 are transparent.

The nipple patch N preferably has ultra-motion function. The ultra-motion function may provide a soft feel and enables the skin to move freely when the nipple patch N is applied to the skin. That is, for the adhesive film 20, the spider film 30, and the finishing film 40, films having the ultra-motion function are preferably used to enable the skin to move freely.

Since the nipple patch N having the ultra-motion function is unable to remain unfolded when removed from the release film 50, the nipple patch N is fell and then folded under the weight thereof.

The nipple patch N being unable to remain unfolded is required to be randomly stretched by force with fingers when attached to the skin around the nipple, which is inconvenient. Nevertheless, the nipple patch N is likely to be attached with curves in random spots.

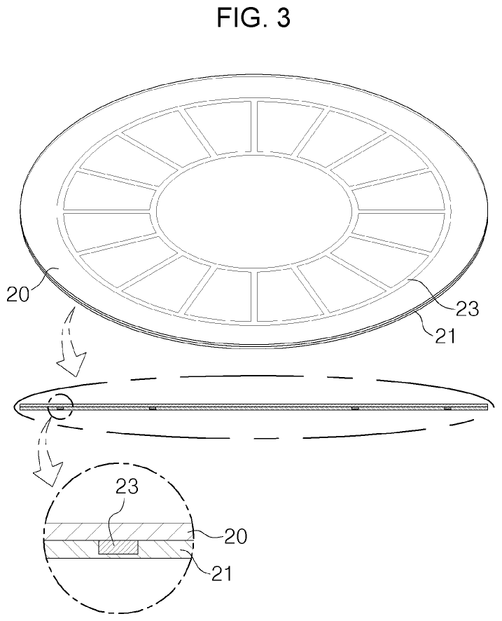

Referring to FIG. 3, in order to solve such problems, the present disclosure provides an anti-folding member 23 included on a frontal surface of the adhesive film 20. As a result, the nipple patch N can remain unfolded when removed from the release film 50. The anti-folding member 23 is covered by the adhesive layer 21, included on the frontal surface of the adhesive film 20, and thus unexposed to the outside.

In this case, when the nipple patch N remains unfolded even after being attached to the skin around the nipple due to the anti-folding member 23, the nipple patch N may insufficiently provide a soft feel, and the skin to which the nipple patch N is attached may be unable to move freely. Therefore, the anti-folding member 23 is made of a material soluble at body temperature so that the anti-folding member 23 is deactivated when the nipple patch N is attached to the skin.

The anti-folding member 23 is made of a material that is a solid at room temperature and melts at about 30° C. to become a liquid. Examples of such a material include gallium, cesium, cocoa butter, and the like.

Referring to FIG. 3, the anti-folding member 23 may be composed of two rings, positioned inward and outward, and connecting lines arranged to connect the two rings.

The anti-folding member 23 may be included on the frontal surface or the rear surface of the adhesive film 20, or the frontal surface of the finishing film 40 to be unexposed to the outside. Preferably, the anti-folding member 23 is included on the frontal surface of the adhesive film 20 so that the material used for the anti-folding member 23 is quickly soluble when the nipple patch N is attached to the skin around the nipple.

Another objective of the present disclosure is to provide the nipple patch remaining unfolded when removed from the release film. As a result, the nipple patch N is easily and quickly attachable without wrinkles and curves when attached to the nipple and skin around the nipple. In addition, the anti-folding member 23 is dissolved once the nipple patch N is completely attached to the skin. Therefore, the nipple patch N is deformable, thereby providing a soft feel and enabling the skin to move freely.

Although in the following description of the preferred embodiments of the present disclosure, the nipple patch in the form of a predetermined form with a predetermined structure has been disclosed with reference to the accompanying drawings, those skilled in the art will appreciate that various alternatives, modifications, additions, and substitutions are possible that may be included within the scope and spirit of the present disclosure as defined by the appended claims.

What is claimed is:

1. A nipple patch comprising:
a shield covering and protecting a nipple; and
a flexible adhesive film having adhesion to skin around the nipple, the adhesive film having a center portion to which the shield is attached,
wherein the nipple patch further comprises a spider film attached to a rear surface of the adhesive film, having lower flexibility than the adhesive film and having an orb web structure to prevent the adhesive film from being stretched.

2. The nipple patch of claim 1, further comprising a finishing film being attached to a rear surface of the spider film and having lower flexibility than the adhesive film to firmly press the nipple.

3. The nipple patch of claim 1, wherein the adhesive film comprises an anti-folding member on a frontal surface thereof and an adhesive layer covering the anti-folding member, the anti-folding member being made of a material soluble at body temperature.
